(19) United States
(12) Patent Application Publication
(10) Pub. No.: US 2026/0139798 A1
(43) Pub. Date: May 21, 2026

(54) Compact, Wall-Mountable LNG Sample Vaporizing Assembly

(71) Applicant: Mustang Sampling, LLC, Ravenswood, WV (US)

(72) Inventors: Kenneth O. Thompson, Ravenswood, WV (US); Nicholas S. Wolfe, Ravenswood, WV (US)

(73) Assignee: Mustang Sampling, LLC, Ravenswood, WV (US)

(21) Appl. No.: 19/121,254

(22) PCT Filed: Nov. 10, 2023
(86) PCT No.: PCT/US2023/037143

§ 371 Date: Apr. 15, 2025

Related U.S. Application Data
(60) Provisional application No. 63/425,018, filed on Nov. 14, 2022.

(51) Int. Cl. F17C 7/04; G01N 1/22; G01N 33/22

(52) U.S. Cl. CPC F17C 7/04; G01N 1/2247; G01N 33/225; F17C 2205/0323; F17C 2205/0338; F17C 2221/033; F17C 2223/0161; F17C 2227/0393; F17C 2250/0439; F17C 2265/066

(57) ABSTRACT

A compact, wall mountable cryogenic LNG sample re-vaporization system and method from a steady state storage source for sample energy content analysis in space constrained environments such as in cruise or cargo ship engine rooms for mounting on a vertical surface with a cabinet containing a controller, having a cryogenic LNG sample input and a vaporized sample output, an input sample temperature detector, a flash vaporizer, a vaporized sample accumulator, a vaporized sample by-pass stream for flow rate measurement, a heated pressure regulator for regulating and conditioning the vaporized sample pressure and an output for communication of the conditioned vaporized sample to an analyzer.

This PCT international application claims priority to U.S. application Ser. No. 63/425,018 filed Nov. 14, 2022.

### TECHNICAL FIELD

The present invention relates to an LNG sample vaporization system particularly suited for utilization when space is limited such as when used in a marine/ship-board engine room. More particularly the invention relates to a reduced size, intelligent re-vaporizing system for sampling cryogenic LNG from a steady state storage tank to be used in powering marine engines.

### BACKGROUND OF THE INVENTION

In marine (ship-board) applications relying on LNG as fuel, systems have been designed and proposed that include relatively complex heat-exchange apparatus to achieve re-vaporization of cryogenic LNG from a storage supply. For example, patents U.S. Pat. Nos. 11,136,103 and 10,823,335 issued to Hyundai Heavy Industries, represent some current thinking about use of heat-exchange from sea water to achieve re-vaporization of LNG for powering an LNG tanker. These overall systems are not readily translatable to sampling and sample conditioning of LNG for analysis in the context of converting to LNG as the fuel for conventional cruise and cargo ships. Furthermore, the descriptions are silent about the need for sampling of the vaporized LNG for analysis of Methane Number or BTU energy content to comply with regulatory or manufacture's operating requirements. Little attention has been dedicated to address the requirements of LNG re-vaporization for sample analysis for the purpose of ensuring effective energy content analysis and record maintenance to meet reporting requirements to maintain a manufacturer's engine warranty or regulations.

A publication entitled Maritime LNG fuel systems for small vessels—A survey of patents, in Transportation Research Part D: Transport and Environment, Volume 119, June 2023, 103766, constitutes a study providing qualitative evaluation of the features of various existing LNG fuel system designs and particularly focuses on the applicability of such systems in medium to small vessels, such as fishing vessels. The study recognizes fuel systems not only need to be operationally robust but also compact to accommodate available space on board such vessels. However, it does not describe any techniques or structures to achieve such goals.

In effect the study underscores the need for smaller sample re-gasification system footprints to conserve space required for on-board placement of a physical re-vaporization facility. It shows that it would be desirable due to the inherent constraints of ship-board architecture, to minimize if not eliminate altogether, an engine room floor space footprint for a sample re-vaporization facility for sample processing. Because space is at a premium in engine rooms and the space limitations imposed by the nature of ordinary cruise and cargo ship essentially equates sacrifice of floorspace to lost revenue from other uses, it is desirable to provide a compact LNG sample conditioning vaporizer system requiring a minimal footprint while providing the necessary components to achieve effective vaporization for energy content validation.

### SUMMARY OF THE INVENTION

It is an object of the present invention to assist users and operators of cryogenic Liquid Natural Gas (LNG) equipment in space challenged environments by providing a sampling vaporizer assembly utilizing minimal space.

It is another object of the invention to provide operation efficiency of conventional full sized sample cryogenic liquid re-vaporization systems including integral functions such as LNG conditioning to maintain pressure, temperature and flow control during the LNG and sample vaporization process.

Still a further object of the invention is to be used in marine applications where engine room space is limited, such as cruise and cargo ships, while allowing for vaporizing and conditioning of LNG samples for analysis.

A further object of this invention is to minimize the space/footprint required for situating a shipboard LNG re-vaporizing system

Another object of the present invention is to provide a complete, compact, intelligent LNG sample re-vaporization system aptly suited for use in space-challenged environments.

Illustrative, non-limiting embodiments of the present invention may overcome some or all the aforementioned and other disadvantages associated with related art LNG vaporization and measurement systems. Also, the present invention is not necessarily required to overcome the disadvantages described above and an illustrative non-limiting embodiment of the present invention may not overcome all the problems described above.

In this regard, the present invention describes a fully functional vaporization unit featuring decreased dimensions while providing comparable performance operability of a full-size sample vaporizing and conditioning system with a wall/bulkhead mounted cabinet assembly that complies with applicable safety requirements for a specific situation (e.g., Flameproof or Class I, Division 1 and even ATEX explosion resistance enclosures, etc.) that effectively enhances utilization of limited on-board space. Because the present invention is directed for use in connection with LNG provided from a steady state source such as a shipboard storage tank rather than a fluctuating source such as a pipeline or for custody transfer or bunkering, the cabinet assembly dispenses with unnecessary components such as filters, a liquid block, an auxiliary cabinet heating unit, etc.

Such space-saving characteristics may also be desirable in non-marine environments where LNG or natural gas liquids are employed as a fuel for power/heating purposes and where extra space is at a premium such as in the case of an Arctic-based research facility.

As described in Applicant's patent U.S. Pat. No. 10,976,295 directed to Methane Number Generation methods, many manufacturers and governmental entities require reporting of LNG energy content to meet warranty and/or regulatory requirements. The present invention also provides for an integrated design using a complement of system components required for achieving archiving measurement and monitoring of the LNG energy content quality (Methane Number and/or BTU value) to meet regulatory and/or engine operating and manufacturer warranty requirements while substantially reducing system footprint and minimizing occupation of valuable floor space.

To achieve the above and other objects an embodiment in accordance with the invention includes a compact system for vaporizing samples of cryogenic Liquid Natural Gas (LNG) used for powering engines in a space limited environment, characterized by: a) a cabinet having a back wall including an element for mounting to a vertical surface, side walls, a top wall, a bottom wall generally defining a rectangular configuration and a front access door, said cabinet side walls having a cryogenic liquid sample input, a vaporized gas sample output and a vaporized gas sample by-pass outlet; b) an electrically powered control unit contained within the cabinet; c) a Resistance Temperature Detector (RTD) electrically connected to the control unit and in fluid communication with the liquid sample input for generating a signal corresponding to the temperature of the liquid sample input; d) an electrically powered flash vaporizer electrically connected to the control unit and in fluid communication with the liquid sample input for receiving liquid sample for flash vaporization; e) an accumulator with an input for receiving and accumulating vaporized sample from the flash vaporizer and an output line for outputting accumulated vaporized sample; and f) an in-line connector associated with the accumulator output line for communicating one stream of the accumulated vaporized sample to an in-line downstream heated pressure regulator with an inlet line and an outlet line to regulate the pressure of the accumulated vapor sample which then passes from an outlet line to the vaporized gas sample output in the cabinet to a downstream analyzer, said connector including a by-pass stream line to direct accumulated vapor sample though an in-line flow meter to measure the flow rate of the accumulated vapor sample and to the cabinet bypass outlet.

The invention provides a further embodiment to the previous embodiment characterized by a shutoff valve disposed in the by-pass line to terminate accumulated vaporized sample flow through the by-pass stream line to the in-line flow meter.

The invention provides a further embodiment to the previous embodiment characterized by a pressure gauge and an in-line shutoff valve disposed between the flash vaporizer and the accumulator to terminate vaporized sample flow to the accumulator.

The invention provides a further embodiment to the previous embodiment further characterized by a pressure gauge and an in-line shutoff valve disposed between the pressure regulator and the vaporized gas sample output to terminate vaporized sample flow to a downstream analyzer.

The invention provides a further embodiment to any of the previous embodiments characterized by the shut-off and flow-control valves are electrically actuated and activated/deactivated by the control unit.

The invention provides a further embodiment to any of the previous embodiments characterized by a pressure relief outlet from the accumulator associated with a bursting disc relief valve.

The invention provides a further embodiment to any of the previous embodiments characterized by the in-line connector associated with the accumulator output line being a T-connector.

Further objects of the invention are met by a method of sample vapor conditioning from a steady state cryogenic natural gas source for energy content sampling with a compact system mounted on a vertical surface including a cabinet with a back wall including a vertical-surface mounting element, side walls, a top wall, a bottom wall generally defining a rectangular configuration and a front access door, a cryogenic liquid sample input, a vaporized gas sample output and a vaporized gas sample by-pass outlet; the cabinet containing an electrically powered control unit connected to a Resistance Temperature Detector (RTD) in fluid communication with the cryogenic liquid sample input for generating a signal corresponding to the temperature of the cryogenic liquid sample input; an electrically powered flash vaporizer electrically connected to the control unit and in fluid communication with the liquid sample input for receiving liquid sample for flash vaporization; an accumulator with an input for receiving and accumulating vaporized sample from the flash vaporizer and an output line for outputting accumulated vaporized sample; and an in-line connector associated with the accumulator output line for communicating one stream of the accumulated vaporized sample to an in-line downstream pressure regulator with an inlet line and an outlet line to regulate the pressure and condition of the accumulated vapor sample passed from the outlet line to the vaporized gas sample output to a downstream analyzer, where the connector includes a by-pass stream line to direct accumulated vapor sample though an in-line flow meter to measure the flow rate of the accumulated vapor sample and connected to the cabinet bypass outlet, the method characterized by the steps of: a) extracting a cryogenic liquid sample from a steady-state liquid natural gas source; b) passing at least a portion of the cryogenic liquid sample through the RTD for generating a signal representative of the cryogenic liquid sample temperature; c) passing the cryogenic liquid sample to a flash vaporizer to generate a vaporized gas sample; d) passing the vaporized gas sample to the accumulator input to mix the accumulated vaporized gas in the accumulator; e) extracting mixed vaporized gas sample from the accumulator through the accumulator output line and passing it to the in-line connector associated with the accumulator output; f) passing at least a portion of the vaporized gas sample to the by-pass stream line to an in-line flow meter to measure the flow rate of the vaporized gas sample to ensure operation within proper operation parameters and then to the cabinet bypass outlet; and g) passing at least a portion of the vaporized sample the in-line downstream pressure regulator and then passing the conditioned vaporized gas sample output to communicate the conditioned vaporized gas sample from the vertical surface mounted cabinet to a downstream analyzer.

The invention provides a further embodiment to the previous method characterized in that the compact system further includes a shutoff valve disposed in the by-pass line and the method further is characterized by the step of actuating the shutoff valve to terminate flow of accumulated vaporized sample through the by-pass stream line to the in-line flow meter.

The invention provides a further embodiment to the previous embodiment characterized in that the compact system further includes a pressure gauge and an in-line shutoff valve disposed between the flash vaporizer and the accumulator, the method further characterized by the step of actuating the shutoff valve to terminate vaporized sample flow to the accumulator.

Still in another embodiment to the previous embodiment the inventive method is characterized in that the compact system includes a pressure gauge and an in-line shutoff valve disposed between the pressure regulator and the vaporized gas sample output where the method includes the step of terminating vaporized sample flow to a downstream analyzer.

The invention provides a further embodiment to any of the previous method embodiments where shut-off valves are electrically actuated, characterized further by the step of activating or deactivating the shut-off valves by the control unit to control flow of vaporized gas sample through the compact system.

In the following description, reference is made to the accompanying drawings, and which is shown by way of illustration to a specific embodiment in which the invention may be practiced. The following illustrated embodiment is described in sufficient detail to enable those skilled in the art to practice the invention. It is to be understood that other embodiments may be utilized and that structural changes based on presently known structural and/or functional equivalents may be made without departing from the scope of the invention.

### BRIEF DESCRIPTION OF THE DRAWINGS

The aspects of the present invention will become more readily apparent by describing in detail the illustrative, non-limiting embodiment thereof with reference to the accompanying drawings, in which:

FIG. 1 is a schematic view of an embodiment of the invention for LNG sample vaporization and conditioning in the form of a wall mountable cabinet.

FIG. 2 is a front view of a physical construct according to FIG. 1 without a front cabinet door.

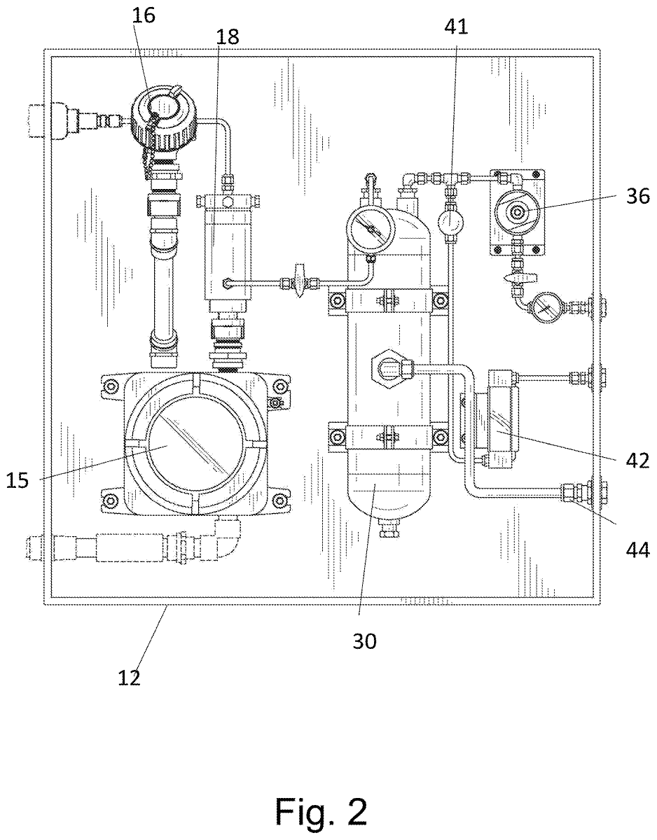

FIG. 3 is a right side view of a physical construct according to FIG. 1 with a handled, closed cabinet door.

### DETAILED DESCRIPTION

An exemplary, non-limiting, embodiment of the present invention is discussed in detail below. While specific configurations and dimensions may be discussed to provide clarity, it should be understood that such disclosed dimensions and configurations are provided for illustration purposes only. A person skilled in the relevant art will recognize that, unless otherwise specified, other dimensions and configurations may be used without departing from the spirit and scope of the invention.

As used herein “substantially”, “relatively”, “generally”, “about”, and “approximately” are relative modifiers intended to indicate permissible variation from the characteristic so modified. They are not intended to be limited to the absolute value or characteristic which it modifies but rather approaching or approximating such a physical or functional characteristic.

In the detailed description, references to “one embodiment”, “an embodiment”, or “in embodiments” mean that the feature being referred to is included in at least one embodiment of the invention. Moreover, separate references to “one embodiment”, “an embodiment”, or “in embodiments” do not necessarily refer to the same embodiment; however, neither are such embodiments mutually exclusive, unless so stated, and except as will be readily apparent to those skilled in the art. Thus, the invention can include any variety of combinations and/or integrations of the embodiments described herein.

The terminology used herein is for the purpose of describing particular embodiments only and is not intended to be limiting of the invention. As used herein, the singular forms, “a”, “an” and “the” are intended to include the plural forms as well, unless the context clearly indicates otherwise. It will be further understood that the root terms “include” and/or “have”, when used in this specification, specify the presence of stated features, integers, steps, operations, elements, and/or components, but do not preclude the presence or addition of at least one other feature, integer, step, operation, element, component, and/or groups thereof.

It will be appreciated that as used herein, the terms “characterized by”, “comprises,” “comprising,” “includes,” “including,” “has,” “having” or any other variation thereof, are intended to cover a non-exclusive inclusion. For example, a process, method, article, or apparatus that is characterized by a list of features is not necessarily limited only to those features but may include other features not expressly listed or inherent to such process, method, article, or apparatus.

It will also be appreciated that as used herein, any reference to a range of values is intended to encompass every value within that range, including the endpoints of said ranges, unless expressly stated to the contrary.

As used herein “connected” includes physical, whether direct or indirect, permanently affixed or adjustably mounted. Thus, unless specified, “connected” is intended to embrace any operationally functional connection.

As used herein “wall-mounted” is intended to describe the system cabinet mounting that is disposed on a wall/bulkhead, above a horizontal support surface such as a floor or deck to preserve limited floor/deck space for dedication to other uses.

In the following description, reference is made to the accompanying drawings which are provided for illustration purposes as representative of the specific exemplary embodiment in which the invention may be practiced. The following illustrated embodiment is described in sufficient detail to enable those skilled in the art to practice the invention. It is to be understood that other embodiments may be utilized and that structural changes based on presently known structural and/or functional equivalents may be made without departing from the scope of the invention.

Given the following detailed description, it should become apparent to the person having ordinary skill in the art that the invention herein provides a novel liquid vaporization device and a method thereof for providing augmented efficiencies while mitigating problems of the prior art.

The embodiment illustrated in the accompanying Figures eliminates the need for the system cabinetry to occupy floor space, in for example, a marine engine room, by possessing a small footprint and allowing for wall mounting above the facility floor.

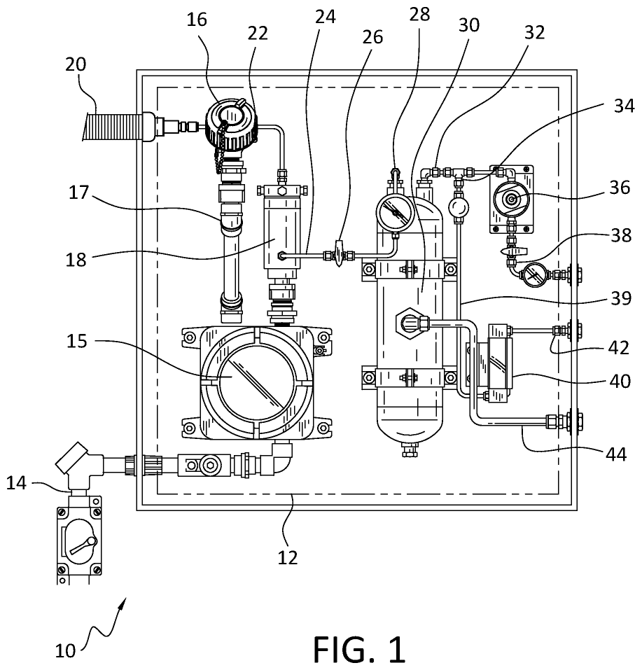

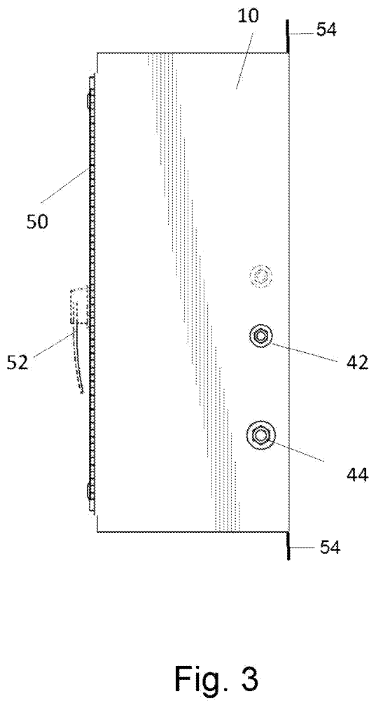

As depicted in FIGS. 1 and 3, vaporizer assembly 10 includes a cabinet housing 12. Cabinet housing 12 is a fireproof container with back, side, top and bottom walls, defining a generally rectangular or square perimeter accessible through a hinged, front cabinet door 50. The cabinet may include a mounting element on the back wall (not illustrated) of known construction for securing to a vertical surface such as a wall or bulk-head. Such mounting element may be in the form of a bracket type mount to cooperate with a complementary rail mounted on the surface to anchor the cabinet 12 thereto or even take the form of simple tabs 54 disposed at the corners with bolt holes for bolting the cabinet to an underlying Unistrut to be immovable relative to the wall. The cabinet is constructed to be fireproof and may even be constructed to be explosion proof. The vaporizer assembly cabinet housing 12 features an electric power inlet 14 to power a controller 15 mounted on the lower portion of the back wall of the cabinet 12. Controller 15 is either a PLC (programmable logic controller) controller or a PID (proportional-integral-derivative) control system used to provide robust control over the connected components, improving response time, and allowing for data logging.

Electrical feeds are provided to a Resistance Temperature Detector (RTD) 16 through electrical conduit 17 and directly to vaporizer 18 from the controller 15. The RTD 16 is programmable which improves accuracy of inlet temperature measurement over conventional thermocouples and minimizes pressure loss through the measurement device. The RTD detects the temperature of the liquid sample from the source input 20 originating from a steady state storage vessel/tank (not illustrated) and generates a signal representative of the temperature transmitted to the controller 15. The RTD is entirely contained within an explosion-proof housing sealed with a screw cover connection head 22.

Downstream of the RTD is an electrically powered LNG vaporizer 18 of conventional/known construction such as the sampling vaporizer structure described in Applicant's patent U.S. Pat. No. 8,056,399 or of Applicant's improved construction described in U.S. Pat. No. 10,613,006, incorporated herein by reference.

The LNG enters the cabinet 12 as a cryogenic liquid through thermally insulating vacuum-jacketed tubing at input 20 through the sidewall. The vacuum-jacketed tubing is of the type described in Applicant's patent U.S. Pat. No. 8,056,399 and provides effective thermal isolation of the cryogenic liquid from the ambient environment. The cryogenic liquid passes through a valved T-connector (not illustrated) where some of the sample is directed to the RTD 16. The cryogenic LNG enters the vaporizer 18 through its top where the liquid is flash vaporized and output through the tube 24 incorporating an in-line shut-off valve 26 (illustrated as a manually controlled valve). The tube 24 includes an in-line pressure gauge and feeds the vaporized gas to entry port 28 disposed at the top of a vapor gas accumulator 30.

The accumulator 30, preferably is of the type described in Applicant's Patent U.S. Pat. No. 7,484,404, incorporated herein by reference, and includes an interior input tube to direct the vapor to an interior location within the accumulator 30 to enhance thorough mixing with gas vapor already present in the accumulator 30 thereby improving the integrity of the accumulated sample for energy content analysis. The accumulator 30 includes relief line that exits the side of the cabinet 12 with a bursting disc to prevent over-pressurization within the accumulator. That relief line may lead to a capture tank or vent to atmosphere.

The thoroughly mixed vapor gas within accumulator tank 30 is removed via additional tubing 32 and passes through T-connector 34 to heated pressure regulator 36 to be output through the vaporized sample output line 38 to an associated analyzer/chromatograph (not illustrated) for sample analysis through the side wall of cabinet 12. The vaporized sample output line 38 includes both an in-line shut-off valve (illustrated as a manually controlled valve) and complementary in-line pressure gauge (illustrated as an analog dial). Descending from the connector 34 is sample by-pass line 39 which includes a flow control valve 41 for controlling or even stopping the flow of mixed vapor gas from the accumulator and an in-line flow meter 40 for monitoring vapor flow rate through the assembly. Where the detected flow rate exceeds proper operation parameters, the excess vapor flow is directed through by-pass line 39 to a by-pass outlet 42 in the side of the cabinet 12. The by-pass outlet 42 may lead to a capture tank or vent to atmosphere.

Selective manipulation of the flow control valve 41, shutoff valve 26, and in-line shut off valve in line 38 may be done manually, or if desirable may be fully automated assuming provision of electrical and signaling connections with the controller 15. Full automation may be achieved by use of electronically controlled solenoid valves which can terminate sample output to an associated analyzer/chromatograph and/or to isolate select segments of the assembly in the event of detected irregularities.

Although only a single embodiment of the invention has been illustrated in the forgoing specification, it is understood by those skilled in the art that many modifications and embodiments of the invention will come to mind to which the invention pertains, having benefit of the teaching presented in the foregoing description and associated drawing. It is therefore understood that the invention is not limited to the specific embodiment disclosed herein, and that many modifications and other embodiments of the invention are intended to be included within the scope of the invention. Moreover, although specific terms are employed herein, they are used only in generic and descriptive sense, and not for the purposes of limiting the description invention.

### INDUSTRIAL APPLICABILITY

The invention is useful for providing a fully functional, compact wall/bulkhead mountable LNG sample re-vaporization and conditioning system for space-challenged environments on a vessel or the like providing for on-board sample energy content analysis from an on-board cryogenic LNG storage tank while substantially reducing system footprint and minimizing occupation of valuable floor space.

1. A compact system for vaporizing samples of cryogenic Liquid Natural Gas (LNG) used for powering engines in a space limited environment, comprising:
a) a cabinet having a back wall including an element for mounting to a vertical surface, side walls, a top wall, a bottom wall generally defining a rectangular configuration and a front access door, said cabinet side walls having a cryogenic liquid sample input, a vaporized gas sample output and a vaporized gas sample by-pass outlet;
b) an electrically powered control unit contained within the cabinet;
c) a Resistance Temperature Detector (RTD) electrically connected to the control unit and in fluid communication with the liquid sample input for generating a signal corresponding to the temperature of the liquid sample input;
d) an electrically powered flash vaporizer electrically connected to the control unit and in fluid communication with the liquid sample input for receiving liquid sample for flash vaporization;
e) an accumulator with an input for receiving and accumulating vaporized sample from the flash vaporizer and an output line for outputting accumulated vaporized sample; and
f) an in-line connector associated with the accumulator output line for communicating one stream of the accumulated vaporized sample to an in-line downstream heated pressure regulator with an inlet line and an outlet line to regulate the pressure of the accumulated vapor sample which then passes from an outlet line to the vaporized gas sample output in the cabinet to a downstream analyzer, said connector including a by-pass stream line to direct accumulated vapor sample though an in-line flow meter to measure the flow rate of the accumulated vapor sample and to the cabinet bypass outlet.

2. The system of claim 1 further comprising a shutoff valve disposed in the by-pass line to terminate accumulated vaporized sample flow through the by-pass stream line to the in-line flow meter.

3. The system of claim 2 further comprising a pressure gauge and an in-line shutoff valve disposed between the flash vaporizer and the accumulator to terminate vaporized sample flow to the accumulator.

4. The system of claim 3 further comprising a pressure gauge and an in-line shutoff valve disposed between the pressure regulator and the vaporized gas sample output to terminate vaporized sample flow to a downstream analyzer.

5. The system of claim 2 where the in-line shut-off valve is electrically actuated and activated/deactivated by the control unit.

6. The system of claim 5 further comprising a pressure relief outlet from the accumulator associated with a bursting disc relief valve.

7. The system of claim 2 further comprising the in-line connector associated with the accumulator output line being a T-connector.

8. A method of sample vapor conditioning from a steady state cryogenic natural gas source for energy content sampling with a compact system mounted on a vertical surface including a cabinet with a back wall including a vertical-surface mounting element, side walls, a top wall, a bottom wall generally defining a rectangular configuration and a front access door, a cryogenic liquid sample input, a vaporized gas sample output and a vaporized gas sample by-pass outlet; the cabinet containing an electrically powered control unit connected to a Resistance Temperature Detector (RTD) in fluid communication with the cryogenic liquid sample input for generating a signal corresponding to the temperature of the cryogenic liquid sample input; an electrically powered flash vaporizer electrically connected to the control unit and in fluid communication with the liquid sample input for receiving liquid sample for flash vaporization; an accumulator with an input for receiving and accumulating vaporized sample from the flash vaporizer and an output line for outputting accumulated vaporized sample; and an in-line connector associated with the accumulator output line for communicating one stream of the accumulated vaporized sample to an in-line downstream pressure regulator with an inlet line and an outlet line to regulate the pressure and condition of the accumulated vapor sample passed from the outlet line to the vaporized gas sample output to a downstream analyzer, where the connector includes a by-pass stream line to direct accumulated vapor sample though an in-line flow meter to measure the flow rate of the accumulated vapor sample and connected to the cabinet bypass outlet, the method comprising the steps of:
a) extracting a cryogenic liquid sample from a steady-state liquid natural gas source;
b) passing at least a portion of the cryogenic liquid sample through the RTD for generating a signal representative of the cryogenic liquid sample temperature;
c) passing the cryogenic liquid sample to a flash vaporizer to generate a vaporized gas sample;
d) passing the vaporized gas sample to the accumulator input to mix the accumulated vaporized gas in the accumulator;
e) extracting mixed vaporized gas sample from the accumulator through the accumulator output line and passing it to the in-line connector associated with the accumulator output;
f) passing at least a portion of the vaporized gas sample to the by-pass stream line to an in-line flow meter to measure the flow rate of the vaporized gas sample to ensure operation within proper operation parameters and then to the cabinet bypass outlet; and
g) passing at least a portion of the vaporized sample the in-line downstream pressure regulator and then passing the conditioned vaporized gas sample output to communicate the conditioned vaporized gas sample from the vertical surface mounted cabinet to a downstream analyzer.

9. The method of claim 8 where the compact system further includes a shutoff valve disposed in the by-pass line and the method further comprising the step of actuating the shutoff valve to terminate flow of accumulated vaporized sample through the by-pass stream line to the in-line flow meter.

10. The method of claim 8 where the compact system further includes a pressure gauge and an in-line shutoff valve disposed between the flash vaporizer and the accumulator, the method further comprising the step of actuating the shutoff valve to terminate vaporized sample flow to the accumulator.

11. The system of claim 3 further comprising a pressure gauge and an in-line shutoff valve disposed between the pressure regulator and the vaporized gas sample output to terminate vaporized sample flow to a downstream analyzer.

12. The method of claim 10 where shut-off valves are electrically actuated, comprising further by the step of activating or deactivating the shut-off valves by the control unit to control flow of vaporized gas sample through the compact system.
